(19) United States
(12) Patent Application Publication
(10) Pub. No.: US 2025/0277776 A1
(43) Pub. Date: Sep. 4, 2025

(54) SAMPLER SYSTEM

(71) Applicant: Amulet, Inc., Madison, WI (US)

(72) Inventors: Jeanette Numbers, Providence, RI (US); Catherine Jameson, Providence, RI (US); Seunghyuk Noh, Cranston, RI (US); Abigail Barnes, Madison, WI (US); Lukasz Mendecki, Worcester, MA (US); Nazir Khan, Cambridge, MA (US)

(73) Assignee: Amulet, Inc., Madison, WI (US)

(21) Appl. No.: 18/712,503

(22) PCT Filed: Nov. 21, 2022
(86) PCT No.: PCT/US2022/050596

§ 371 Date: May 22, 2024

Related U.S. Application Data
(60) Provisional application No. 63/282,057, filed on Nov. 22, 2021.

(51) Int. Cl. G01N 33/02; G01N 1/28; G01N 27/327

(52) U.S. Cl. CPC G01N 33/02; G01N 1/286; G01N 27/327; G01N 2001/2866

(57) ABSTRACT

A device for detecting an ingredient in a food sample includes a cap having a first piercer member and a collar that receives the food sample and couples to the cap. The collar includes a first frangible barrier and a grinding assembly, which includes a second piercer member, disposed between the first frangible barrier and a second frangible barrier spaced therefrom. The collar further includes a sensor disposed below the second frangible barrier. The first and second frangible barriers form a first chamber containing a buffer solution, and the second frangible barrier and the sensor form a second chamber. Responsive to coupling the cap and collar, the first piercer member pierces the first frangible barrier and the second piercer member pierces the second frangible barrier. After the first and second frangible barriers are pierced, the food sample passes through the first and second chambers to be received by the sensor.

### CROSS-REFERENCE TO RELATED APPLICATIONS

This application claims the benefit of U.S. Provisional Application No. 63/282,057, filed Nov. 22, 2021, the contents of which are incorporated herein by reference in their entirety.

### TECHNICAL FIELD

The present disclosure relates generally to the field of detection services and devices. More specifically, the present disclosure relates to a system and method for detecting an ingredient within a food sample.

### BACKGROUND

Food can include various ingredients that may cause irritation, harm, or even death of consumers. Often, consumers are unaware or unable to determine whether such ingredients are present in their food. Although some foods may be labeled as containing one or more ingredients known to cause allergic reactions or other severe immune responses, not all foods or food products are labeled or labeled appropriately, which poses serious risks to consumers.

### SUMMARY

One aspect of the present disclosure relates to a device for detecting an ingredient in a food sample. The device includes a cap having a first piercer member, and a collar configured to receive the food sample and configured to couple to the cap. The collar includes a first frangible barrier extending across a first cross section of the collar and a grinding assembly disposed between the first frangible barrier and a second frangible barrier. The grinding assembly includes a second piercer member, where the second frangible barrier extends across a second cross section of the collar at distance below the first cross section. The collar further includes a sensor disposed below the second frangible barrier. A first chamber is formed between the first frangible barrier and the second frangible barrier, the first chamber containing a volume of a buffer solution. In any embodiment herein, the sensor may be an sensor. A second chamber is formed between the second frangible barrier and the sensor. In response to the cap being coupled to the collar, the first piercer member is configured to pierce the first frangible barrier and the second piercer member is configured to pierce the second frangible barrier. In response to the each of the first and second frangible barriers being pierced, the first chamber becomes fluidly coupled to the second chamber, and the food sample received by the collar passes through each of the first chamber and the second chamber to be received by the sensor.

In various embodiments, the collar is threadably coupled to the cap. In some embodiments, the grinding assembly is disposed above the first chamber, where the food sample mixes with the buffer solution within the first chamber. In other embodiments, the grinding assembly includes a support member, which is configured to concentrically fit within the first chamber such that an outer perimeter defined by an upper surface of the support member is adjacent an inner surface of the first chamber. In yet other embodiments, the support member forms a boss, which is centrally disposed within the support member and aligned with a longitudinal axis of the device. The boss includes a bore defined therein, and wherein the second piercer member is configured to fit within and articulate relative to the bore of the central boss. In various embodiments, the boss projects downward toward a bottom surface of the device and away from the upper surface of the support member. In some embodiments, the grinding assembly includes at least two spikes, the at least two spikes projecting upward from the upper surface of the support member. In other embodiments, the support member includes a plurality of apertures disposed within the upper surface, where the collar includes a receptacle configured to receive the food sample. The receptacle is defined between an upper edge of the collar and the first frangible barrier, and the plurality of apertures enable fluid communication between the receptacle and the first chamber.

In various embodiments, the first piercer member extends from a surface within the cap toward the collar. In some embodiments, the first piercer member has a first axis and the second piercer has a second axis, where the first axis is aligned with the second axis when the cap is coupled to the collar. In other embodiments, the second piercer member has a first end and a second end opposite the first end, the first end extending toward the first frangible barrier and the second end extending toward the second frangible barrier. The first piercer member is configured to contact the first end of the second piercer member upon piercing the first foil. The first piercer member contacting the second piercer member causes the second piercer member to pierce the second frangible barrier. In yet other embodiments, the second piercer member slides within the first chamber to pierce the second frangible barrier responsive to the first piercer member contacting the first end of the second piercer member.

Another aspect of the present disclosure relates to a system for detecting an ingredient in a food sample. The system includes a sampler unit configured to receive the food sample. The sampler unit includes a first piercer member, a first frangible barrier extending across a first cross section of the sampler unit, and a first grinding assembly disposed between the first frangible barrier and a second frangible barrier. The first grinding assembly includes a second piercer member, where the second frangible barrier extends across a second cross section of the sampler unit at distance below the first cross section. The sampler unit also includes an sensor disposed below the second frangible barrier. The system further includes an analysis unit configured to be received within a portion of the sampler unit, where the analysis unit includes a processor, and where the processor is communicatively couplable to the sensor. A first chamber is formed between the first frangible barrier and the second frangible barrier. A second chamber is formed between the second frangible barrier and the sensor. The first piercer member is configured to pierce the first frangible barrier and the second piercer member is configured to pierce the second frangible barrier. In response to the each of the first and second frangible barriers being pierced, the first chamber becomes fluidly coupled to the second chamber. The food sample received within the sampler unit passes through each of the first chamber and the second chamber to be received by the sensor. The processor is configured to determine a presence of the ingredient in the food sample.

In various embodiments, the second piercer member includes a first end and a second end opposite the first end, where the first piercer member is configured to contact the first end of the second piercer member when the sampler unit is assembled. In some embodiments, the second piercer member is configured to articulate within the grinding mechanism responsive to being contacted by the first piercer member such the second piercer moves from a first position to a second position, where the second end of the piercer is disposed above the second frangible barrier when in the first position, and where the second end of the piercer extends below the second cross-section. In other embodiments, the sampler unit includes a cap and a collar couplable to the cap. The cap includes the first piercer member and the collar includes the second piercer member. In yet other embodiments, the cap further includes a second grinding assembly, where the second grinding assembly is configured to engage with the first grinding assembly when the collar is coupled to the cap.

Yet another aspect of the present disclosure relates to method for detecting an ingredient in a food sample. The method includes placing the food sample within a collar and coupling the collar to a cap. A first piercer member disposed within the cap pierces a first frangible barrier within the collar responsive to coupling the collar to the cap, and a second piercer member axially stacked with the first piercer member is configured to pierce a second frangible barrier within the collar responsive to the first piercer member piercing the first frangible barrier. The method further includes coupling an analysis unit to the collar by inserting an end of the analysis unit within a recess of the collar, where the analysis unit comprises a processor. The processor is communicatively couplable to an sensor disposed within the collar, and the sensor is in fluid communication with at least a portion of the food sample responsive to the second piercer member piercing the second frangible barrier. The method also includes determining a presence of the ingredient in the food sample based on an output by the processor within the analysis unit.

In various embodiments, the ingredient includes a food ingredient. In some embodiments, the food ingredient includes at least one of an allergen, food toxin, or food pathogen.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a side perspective view of a sampler unit, according to an exemplary embodiment.

FIG. 2 is a side perspective view of a sampler unit, according to another exemplary embodiment.

FIG. 3 is an end perspective view of the sampler unit of FIG. 1, according to an exemplary embodiment.

FIG. 4 is an exploded perspective view of the sampler unit of FIG. 1 oriented to show a top end of the sampler unit, according to an exemplary embodiment.

FIG. 5 is an alternate exploded perspective view of the sampler unit of FIG. 1 oriented to show a bottom end of the sampler unit, according to an exemplary embodiment.

FIG. 6 is a partially exploded side cross-sectional view of the sampler unit of FIG. 1 taken along line 6-6, according to an exemplary embodiment.

FIG. 7 is a side cross-sectional view of a chamber within the sampler unit of FIG. 6, according to an exemplary embodiment.

FIG. 8 is a side cross-sectional view of an electrode assembly within the sampler unit of FIG. 6, according to an exemplary embodiment.

FIGS. 9-12 show side cross-sectional views of the sampler unit of FIG. 1 in varying stages of operation, according to various exemplary embodiments.

FIGS. 13-16 show side perspective views of a sampler system including the sampler unit of FIG. 1 in varying stages of operation, according to various exemplary embodiments.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting

Referring generally to the figures, the present technology disclosure relates to a fast and portable sampler unit and system that enables a user to directly sample tangible goods (e.g., foods) to determine the presence of one or more molecules (e.g., allergens, pathogens, and/or toxins). For example, the sampler unit may provide individuals with information about the contents and relative safety of their food and other tangible goods. The presence or absence of the molecule may be detected by capturing a representative sample, such as a liquid or solid goods sample (i.e., from a portion of food) and exposing the sample to a sensor. The sensor may then be connected to an analysis unit (e.g., Amulet), which includes one or more processing devices. The one or more processing devices may then detect a measurable amount of the one or more molecules and subsequently alert the user.

In various embodiments, at least one of the sampler unit or the analysis unit can be configured as a wearable device, or integrated into everyday products that users may keep on their person (e.g., cellular phone, watch, keychain, necklace, wearable fitness monitors, etc.). A software application (i.e. “app”), may accompany the processing device, where users may track and upload tests, connect with other users, and store and share important information including, but not limited to, emergency contacts.

The following terms are used throughout this disclosure, as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Unless otherwise indicated, numeric ranges, for instance as in “from 2 to 10,” are inclusive of the numbers defining the range (e.g., 2 and 10).

Unless otherwise indicated, ratios, percentages, parts, and the like are by weight.

In any embodiment, the molecule in which the sensor is configured to detect may include an allergen, a trace molecule of an allergen, a pathogen, a toxin, or a combination of any two or more thereof.

As used herein “allergen” refers to both allergy and intolerant inducing substances. A true allergy causes an immune system reaction that affects numerous organs in the body and can cause a range of symptoms. In some cases, an allergic reaction can be severe or life-threatening. In comparison, intolerance symptoms are generally less serious and often limited to digestive problems. Nonlimiting examples of intolerances include absence of an enzyme needed to fully digest a consumable (e.g., food or drink), irritable bowel syndrome, sensitivity to an additive, recurring stress or psychological factors, and celiac disease. An example of an absence of an enzyme is lactose intolerance. Irritable bowel syndrome is a chronic condition that may cause cramping, constipation, and/or diarrhea. An example of sensitivity to an additive are sulfites commonly used to preserve food and drinks. Celiac disease has some features of a true food allergy because it involves the immune system, however, symptoms are mostly gastrointestinal, and people with celiac disease are not at risk of anaphylaxis.

Allergens may include, but are not limited to, animal products, grains (e.g., gluten), vegetables, fruits, dairy products, fish, beverages, legumes, chocolates, synthetic food chemicals (e.g., monosodium glutamate (MSG), artificial sugars such as aspartame), and any combinations of two or more thereof. In one example, an allergen may include a food protein. In any embodiment, the allergen or the trace molecule may be a peanut allergen, tree nut allergen, milk allergen, egg allergen, wheat allergen, soy allergen, meat allergen, fish allergen, shellfish allergen, coconut allergen, or a combination of two or more thereof. In any embodiment, the allergen or the trace molecule may be a nut allergen listed in Table 1. In any embodiment, the allergen or the trace molecule may be a tree nut allergen (e.g., almond, almond paste, or a combination thereof). In any embodiment, the allergen or the trace molecule may be a soy allergen. In any embodiment, the allergen or the trace molecule may include a flavonoid, amygdalin, or a combination thereof. In any embodiment, the flavonoid may include an isoflavonoid, neoflavonoid, or derivatives thereof. In any embodiment, the isoflavonoid or derivative thereof may include isoflavones, isoflavonones, isoflavans, pterocarpans, rotenoids, or combinations of two or more thereof. In any embodiment, the allergen or the trace molecule may include amygdalin, apigenin-6-arabinoside-8-glucoside,apigenin-6-glucoside-8-arabinoside, arachin, biochanin A, catechin gallate, crysoeriol, cyanocobalamin, daidzein, daidzin,5-5′-dehydrodiferulic acid, 5-8′-dehydrodiferulic acid, 5,7-dihydroxychromone, 5,7, dimethoxyisoflavone, ferulic acid, galactose, genistein, genistin, 3-hydroxybiochanin A, isochlorogenic acid, isoferulic acid, juglone, lactose, lariciresinol, medioresinol, procyanidin B2, procyanidin C1, resveratrol, resveratrol 3-glucoside, secoisolariciresinol, syringaresinol, syringic acid, trans-sinapic acid.

As used herein a “trace molecule of an allergen” refers to molecules that are suitable for detecting the presence of an allergen but may not necessarily be allergens themselves. In any embodiment, the trace molecule of the allergen may be an organic molecule or a salt thereof. For example, the trace molecule may be the allergen itself, epitope of an allergen (i.e., the part of an antigen molecule to which an antibody attaches itself), molecule that is commonly present with an allergen, a subunit of an allergen, a derivative of an allergen, or a combination of two or more thereof including a polypeptide, protein, epitope, aptamer, or a combination of any two or more thereof. In some embodiments, the organic molecule may include at least one protein. In some embodiments, the organic molecule may include at least two different proteins. In some embodiments, the organic molecule may include at least one epitope. In some embodiments, the organic molecule may include at least two different epitopes. In some embodiments, the organic molecule may include at least one protein and at least one epitope. In some embodiments, the organic molecule may be selected from lactose, galactose, amygdalin, juglone, biochanin A, resveratrol daidzein, daidzin, genistein, genistin, and a combination of any two or more thereof. In any embodiment, the organic molecule may not include cortisol, an amino acid, theophylline, and/or chlorpyrifos.

Pathogens may include, but are not limited to, a bacterium, virus, or other microorganism that can cause disease and/or illness. In any embodiment, the pathogen may be a food pathogen and/or a clinical pathogen. Exemplary food pathogens include, but are not limited to, Campylobacter, Cyclospora, Clostridium botulinum, Escherichia coli, Listeria, Salmonella, Staphylococcus aureus, Shigella, Toxoplasma gondii, Vibrio vulnificus, Norovirus, Hepatitis A, or a combination of any two or more thereof. Exemplary clinical pathogens include, but are not limited to, Candida, Chlamydia trachomatis, Neisseria gonorrhoeae, Methicillin-resistant Staphylococcus aureus, Mycobacterium tuberculosis, human papillomavirus (HPV), Hepatitis B, Hepatitis C, Hepatitis D, Hepatitis E, human immunodeficiency virus, influenza, or a combination of any two or more thereof.

Toxins may include, but are not limited to, herbicides, pesticides, drugs of abuse, or a combination of any two or more thereof. As used herein, herbicides refers to substances that are toxic to plants and commonly used to destroy unwanted vegetation. As used herein, herbicides refers to substances that are toxic to insects or other organisms harmful to cultivated plants or to animals. Exemplary herbicides and/or pesticides include atrazine, azinphos-methyl, bentazone, carbaryl, carbofuran, chlorpyrifos methyl, chlorsulfuron, cyhexatin, diazinon, dimethoate, fenobucarb, glyphosate, hydrazine, imidacloprid, lindane, methyl parathion, paraquat, parathion, permethrin, pirimicard, sulfentrazone, or a combination of any two or more thereof. As used herein, drugs of abuse refers to illegal drugs as well as prescription or over-the-counter drugs that are used for purposes other than those for which they are meant to be used, or in excessive amounts. Exemplary drugs of abuse include an amphetamine or a metabolite thereof (e.g., methamphetamine, 3,4-methylenedioxyamphetamine (MDA), phentermine, ephedrine, and/or pseudoephedrine), cocaine or a metabolite thereof (e.g., benzoylecgonine), a benzodiazepine or a metabolite thereof (e.g., diazepam, temazepam, chlordiazepoxide, nordiazepam, oxazepam, α-hydroxyalprazolam, α-hydroxytriazolam, 7-aminoclonazepam, 7-aminoflunitrazepam, and/or hydroxyethyl-flurazepam), a barbiturate or a metabolite thereof (e.g., mephobarbital. Phenobarbital, butalbital, amobarbital, pentobarbital, and/or secobarbital), a dissociative drug or a metabolite thereof (e.g., ketamine and/or norketamine), an opioid or a metabolite thereof (e.g., fentanyl, norfentanyl, methadone, 2-ethylidene-1,5-dimethyl-3,3-diphenylpyrrolidine (EDDP), buprenorphine, norbuprenorphine, diacetylmorphine (heroin), 6-monoacetylmorphine (6-MAM), morphine, codeine, hydrocodone, hydrocodone, norhydrocodone, dihydrocodeine, hydromorphone, oxycodone, oxymorphone, naloxone, noroxymorphone, and/or noroxycodone), or a combination of any two or more thereof.

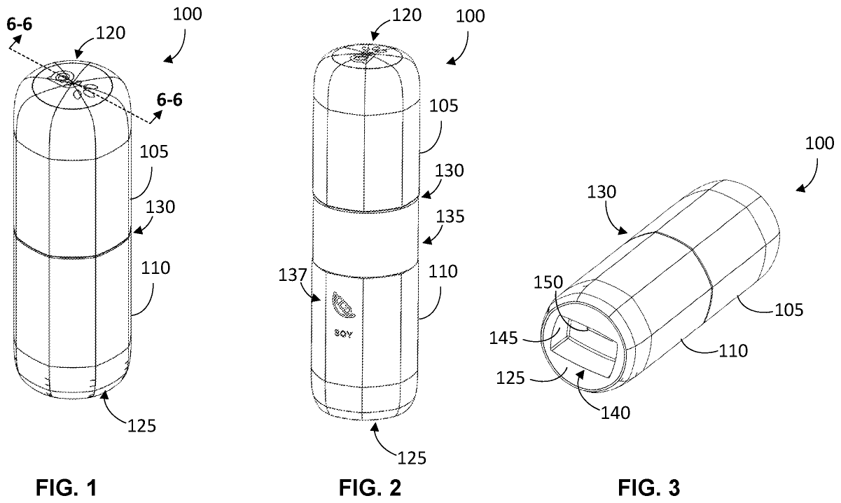

Turning now to the figures and referring specifically to FIG. 1, a sampler unit 100 for detecting one or more molecules (i.e., an ingredient) from within a solid, liquid, or manufactured good (e.g., food) is shown, according to an exemplary embodiment. The ingredient may include an allergen, a toxin, a pathogen, or any other constituent present within the manufactured good. The sampler unit (“device”) 100 includes a first portion (“cap”) 105 and a second portion (“collar”), which is couplable to the cap 105. As shown, the cap 105 forms a first end 120 of the device 100 and the collar 110 defines a second end 125 of the device 100. In various embodiments, the cap 105 abuts the collar 110 at an interface 130 when coupled together. In other embodiments, such as shown in FIG. 2, the collar 110 includes a cuff 135 disposed within an upper portion of the collar 110, where the interface 130 is formed between the cap 105 and cuff 135 of the collar 110. In various embodiments, the cuff 135 is configured to prevent engagement between the cap 105 and the collar 110. The cuff 135 may be removably coupled to the collar 110, where the cuff 135 may be decoupled from the collar 110 to allow the cap 105 to fully engage with the collar 110. In other embodiments, the cuff 135 may be configured to displace (e.g., slide, rotate, etc.) relative to the collar 110 such that the cuff 135 may be repositionable to allow engagement between the cap 105 and the collar 110. In some embodiments, the device 100 may include one or more visual or haptic indicia 137, which may indicate to a user of the device 100 what molecule type the device is configured to detect. As illustrated in FIG. 3, the device 100 includes a receiving port 140, disposed within the second end 125. The port 140 may be configured to receive one or more processing devices, which may facilitate detection of the one or more molecules within the good. The port 130 is defined by a first recess 145 and a second recess 150 extending from the first recess 145.

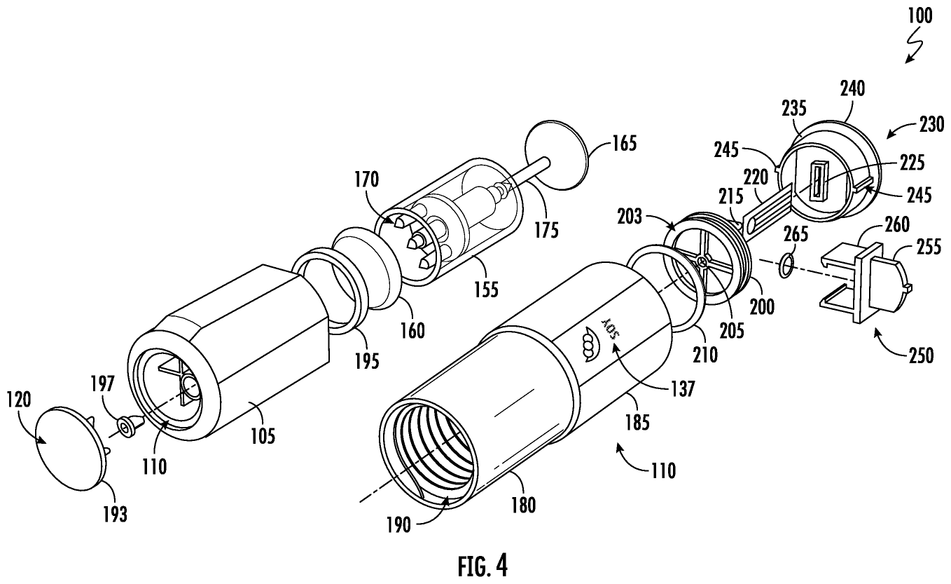

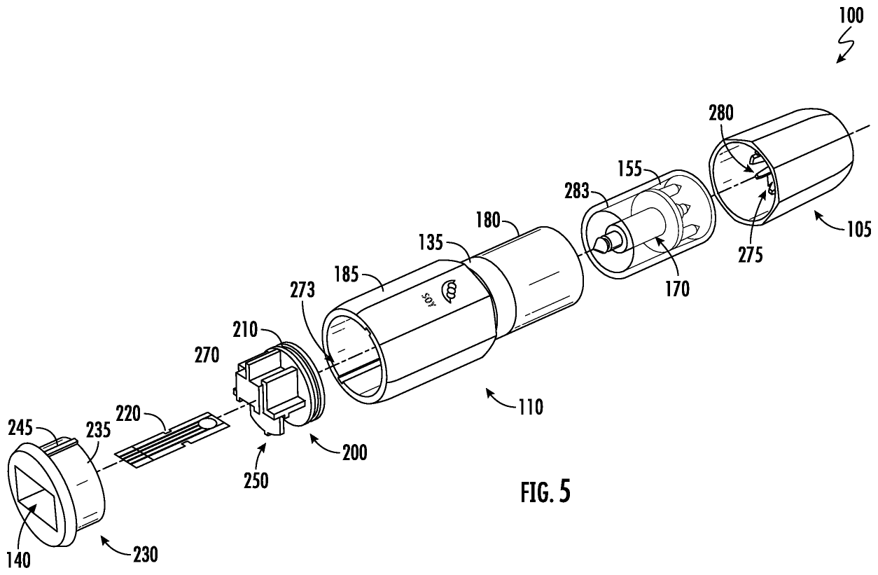

Referring now to FIGS. 4 and 5, the device 100 includes a chamber 155, which is defined between a first frangible barrier (e.g., foil, polymeric membrane) 160 extending across a first section (i.e., a first cross-section) of the device 100 and a second frangible barrier (e.g., foil, polymeric membrane) 165 extending across a second section (i.e., a second cross-section) of the device 100, the second section being spaced from the first section. Each of the first and second frangible barriers 160, 165 are coupled to opposing ends of the chamber 155. In particular, an outer perimeter of each of the barriers 160, 165 may be coupled to the uppermost surface of each end of the chamber 155. In various embodiments, the barriers 160, 165 may be coupled to the chamber 155 via heat sealing, sonic welding, one or more adhesives, and/or any other suitable coupling method know in the art.

As shown, the chamber 155 houses a grinding assembly 170, which is generally axially aligned with the chamber 155. The grinding assembly 170 defines a grinding interface or surface, which facilitates break down of the one or more goods (e.g., food) inserted into the sampler unit 100. The grinding assembly 170 includes a piercer member 175, which articulates within the grinding assembly 170 relative to the chamber 155 to pierce the frangible barrier 165 when the device 100 is activated. In various embodiments, the chamber 155 contains a volume of buffer solution, the buffer solution formulated to mix with the good (i.e., food) inserted into the device 100 as said good is being ground by the grinding assembly 170. In various embodiments, the buffer solution may include water, aqueous buffer, an electrolyte solution, an organic solvent (e.g., ethanol), or a combination of two or more thereof. In any embodiment, the aqueous buffer may include a mild alkaline buffer solution (pH ˜9-11 carbonate/bicarbonate). In any embodiment, the solvent may include an electrolyte solution (e.g., potassium chloride solution). The frangible barriers 160, 165 are configured to fluidly seal the chamber 155 until at least one of the barriers 160, 165 is pierced.

The chamber 155, along with the grinding assembly 170 and the barriers 160, 165 are housed within a first section 180 of the collar 110. As shown, the collar 110 includes the first section 180, which is disposed above and is contiguous with a second section 185, where the first section 180 has a smaller outer diameter compared to the second section 185. In various embodiments, the smaller diameter of the first section 180 relative to the second section 185 allows for the cap 105 to overlap with the collar 110 when the cap 105 is coupled to the collar 110 (i.e., when the device 100 is activated). The sections 180, 185 include a central bore 190, which is configured to house the chamber 155 when the device 100 is assembled and activated.

As shown, the device 100 includes one or more sealing members, which are configured to fluidly seal the device 100 to prevent leakage of the good, the buffer solution, or the mixture thereof from the device 100. In various embodiments, the cap 105 includes a crown 193, which is removably couplable to the first end 120 of the cap 105 via one or more fasteners and is fluidly sealed by one or more sealing members 197 (e.g., gaskets, washers, o-rings, etc.). Alternatively or in addition, the cap 105 may include one or more sealing members 195 (e.g., gaskets, washers, o-rings, etc.) disposed at an end of the cap 105 configured to engage with the collar 110 (i.e., an end of the cap 105 opposite the end 120).

The device 100 also includes a second chamber formed by a repository 200 and defined between the frangible barrier 165 and an electrode 220. The repository 200 is disposed adjacent to the first chamber 155 in a position below the first chamber 155 relative to the first end 120 of the device 100. The repository 200 is fluidly sealed within the collar 110 via one or more sealing members 210 (e.g., gasket, o-ring, etc.). As shown, the repository may be configured to extend across a section of the collar 110 such that an outer perimeter of at least a portion of the repository 200 (or of the one or more sealing members 210) interfaces or engages with an inner surface of the collar 110. The second chamber formed by the repository 200 is configured to be fluidly coupled to the first chamber 155 when the frangible barrier 165 is pierced (i.e., by the piercer member 175). The repository 200 includes a receptacle (“dish”) 203 formed within an upper portion of the repository 200. The dish 203 is configured to receive the good and buffer solution mixture from the first chamber 155 responsive to the frangible barrier 165 being pierced. An opening 205 disposed within the dish 203 subsequently receives the mixture and routes said mixture through a fluid pathway 215 to the electrode 220. The electrode 220 then senses the mixture for determination of the presence of the one or more molecules within the good.

In various embodiments, an end of the electrode 220 is coupled to or in fluid communication with the fluid pathway 215. As shown, the device 100 may include a removable clip 250, which couples to the electrode 220 and/or the repository 200 (e.g., via the fluid pathway 215) to retain the electrode 220 in fluid communication with the repository 200. The clip 250 may include two opposing flanges 260, which are coupled to a tab 255, where the flanges 260 are configured to couple to opposing sides of the electrode 220 and/or the repository 200 to prevent separation of the electrode 220 from the repository 200. The tab 255 may facilitate placement and/or retention of the clip 250 within the collar 110. In various embodiments, the device 100 may include one or more sealing members 265 (e.g., gasket, o-ring) to fluidly seal the coupling between the fluid pathway 215 and the electrode 220.

The electrode 220 is held within the collar 110 via a cover 230, which is removably couplable to the end 125 of the collar 110. The cover 230 includes a recess 225, which is configured to receive an end of the electrode 220 to secure the electrode 220 within the device 100. As shown, the cover 230 includes a first section 235 and a contiguous second section 240, where the first section 235 has a smaller outer diameter compared to the second section 240 to facilitate a friction fit or press fit coupling of the cover 230 within the collar 110. In various embodiments, the cover 230 may also include circumferentially arranged ridges 245, which may facilitate retention of the cover 230 within the collar 110 (e.g., via an interference fit) and/or prevent rotation of the cover 230 (and the electrode 220) relative to the collar 110. In various embodiments, the collar 110 may include one or more protrusions or ridges 273 disposed along an inner surface, which may engage with the ridges 245 of the cover 230 to prevent axial rotation thereof. In addition, as illustrated in FIG. 5, the repository 200 may include one or more fixtures (e.g., slots, recesses, etc.) 270, which may be configured to facilitate retention of the electrode 220 within the device 100.

Activation of the device 100 begins with coupling of the cap 105 to the collar 110. As shown in FIG. 5, the cap 105 includes a grinding assembly 275, which forms a grinding interface to facilitate breaking up the good (i.e., food) inserted into the device 100. The grinding assembly 275 includes a piercer member 275. The piercer member 275 extends from the cap 105 toward the collar 110 such that when the cap 105 is coupled to the collar 110, the piercer member 275 may pierce the frangible barrier 160 and contact the piercer member 175, which then may articulate relative to the grinding assembly 170 to pierce the frangible barrier 165. Piercing of the frangible barriers 160, 165 enables fluid communication among the first chamber 155, the second chamber formed by the repository 200, and the electrode 220, which facilitates detection of the one or more molecules within the good inserted into the device 100.

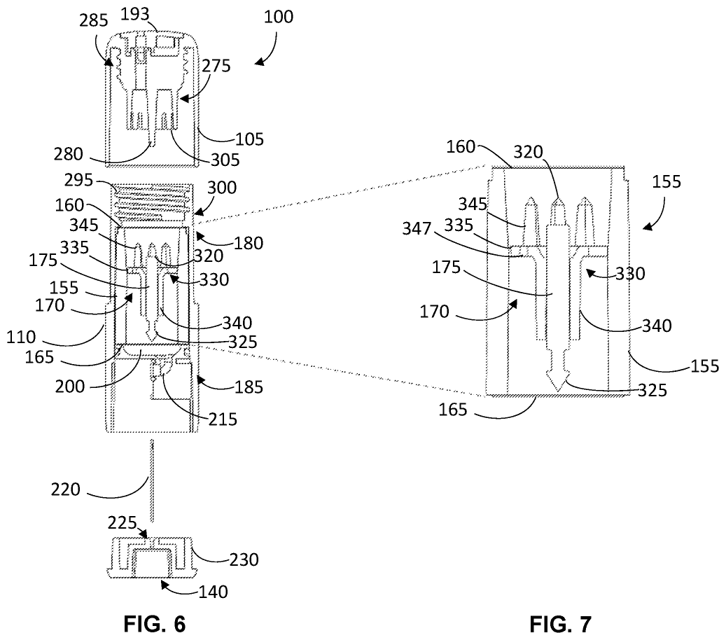

To facilitate molecular detection, the device 100 is configured to break down the good (i.e., food) inserted into the device 100, mix the ground good with the buffer solution within the chamber 155, and then pass the mixture to the electrode 220. FIG. 6 shows a cross-sectional view of the device 100, taken along line 6-6 of FIG. 1. To use the device 100, a good, such as a food, may be inserted into a receptacle 300 formed within the first section 180 of the collar 110. Prior to coupling the cap 105 to the collar 110, the inserted good may rest atop the frangible barrier 160.

To activate the device 100 and initiate detection of one or more molecules within the inserted good, the cap 105 is coupled to the first section 180 of the collar 110. As shown in FIG. 6, the cap 105 includes the grinding assembly 275 disposed within an end of the cap 105. The grinding assembly 275 may include a threaded portion 285, which may be configured to threadably couple to a corresponding threaded portion 295 of the collar 110. As shown, the grinding assembly 275 includes the piercer member 280, which extends away from the end of the cap 105 and toward the collar 110. The grinding assembly 275 also includes one or more protrusions (e.g., spikes) 305, which project away from the end of the cap 105 toward the collar 110. In various embodiments, the protrusions 305 may be disposed in a substantially circular arrangement within the grinding assembly 275. As shown, the grinding assembly 275 is configured such that the piercer member 280 has a length greater than each of the protrusions 305.

When the cap 105 is coupled to the collar 110, the piercer member 280 pierces the frangible barrier 160, which allows the good within the receptacle 300 to enter the chamber 155. In embodiments where the device 100 includes the cuff 135, the cuff 135 acts as a barrier to prevent the cap 105 from fully engaging with the collar 110 to thus prevent the piercer member 280 from piercing the frangible barrier 160. Accordingly, a user of the device 100 may remove or displace the cuff 135 prior to inserting a good (e.g., food sample) into the device 100. As the cap 105 is further threaded onto the collar 110, the grinding assembly 275 engages with the grinding assembly 170. Accordingly, the good within the chamber 155 is ground between the grinding interface formed by the grinding assembly 275 and the grinding interface formed by the grinding assembly 170. As shown, the grinding assembly 170 includes a support member 330, which is configured to concentrically fit within the chamber 155 such that an outer perimeter defined by an upper portion or surface 335 of the support member 330 is adjacent to or abuts against an inner surface of the chamber 155. The piercer member 175 is configured to fit within the support member 330 such a first end 320 of the piercer member 175 extends above the upper portion 335 of the support member 330 and a second end 325 of the piercer member 175 projects below the upper portion 335 of the support member 330. In various embodiments, the support member 330 forms a boss 340, which is centrally disposed within the support member 330 and aligned with a longitudinal axis of the device 100, where the piercer member 175 is configured to fit within and articulate relative to a bore of the boss 340.

As shown, the first end 320 of the piercer member 175 extends upward toward the cap 105. The piercer member 175, which extends along a first axis, and the piercer member 280, which extends along a second axis, may be axially aligned such that when the cap 105 is coupled to the collar 110, a terminal end of the piercer member 180 contacts and applies an axial force to the first end 320 of the piercer member 175. Responsive to the force applied by the piercer member 280, the piercer member 175 may articulate (e.g., slide) relative to the support member 330 in a downward direction such that the second end of the piercer member 175 pierces the frangible barrier 165.

As force is being applied to the piercer member 175 by the piercer member 280 while the cap 105 is being coupled (i.e., threaded) to the collar 110, the grinding assembly 275 articulates relative to the grinding assembly 170. As illustrated in FIG. 7, the grinding assembly 170 includes one or more protrusions (e.g., spikes) 345, which extend away from the upper portion 335 of the support member 330 and toward the cap 105. Accordingly, when the good inserted into the device 100 enters the chamber 155 responsive to the frangible barrier 160 being pierced (i.e., when the cap 105 is coupled to the collar 110), the protrusions 305 of the grinding assembly 305 grind the good against the protrusions 345 of the grinding assembly 170. In various embodiments, the one or more protrusions 305 and/or 345 may be substantially conical in shape. In other embodiments, the protrusions 305, 345 may be cylindrical, rectangular, or a combination thereof. In yet other embodiments, the protrusions 305, 345 may be tapered or shaped as obelisks.

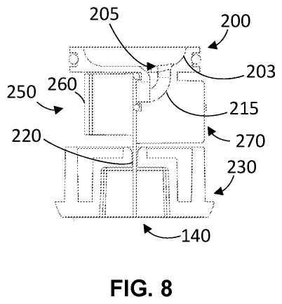

To facilitate fluid communication between the chamber 155 and the repository 200, the support member 330 may include a plurality of apertures (e.g., slots, holes, etc.) 347 disposed through the upper portion 335. Accordingly, the mixture of buffer solution and ground good within the chamber 155 may pass through the apertures 347 and into the repository 200 when the frangible barrier 165 is pierced responsive to the cap 105 and collar 110 being coupled together. Once the frangible barrier 165 is pierced, the mixture may flow from the chamber 155 into the dish 203 of the repository 200, which is shown in FIG. 8. As illustrated, the fluid pathway 215 may receive the mixture via the opening 205.

Activation of the device 100 for determination of one or molecules within the good inserted into the device is initiated when the piercer member 280 causes the piercer member 170 to displace from a first position, where the second end 325 of the piercer member 170 is disposed above the frangible barrier 165, and a second position, where the second end 325 has pierced the frangible barrier 165 and is consequently disposed therebelow.

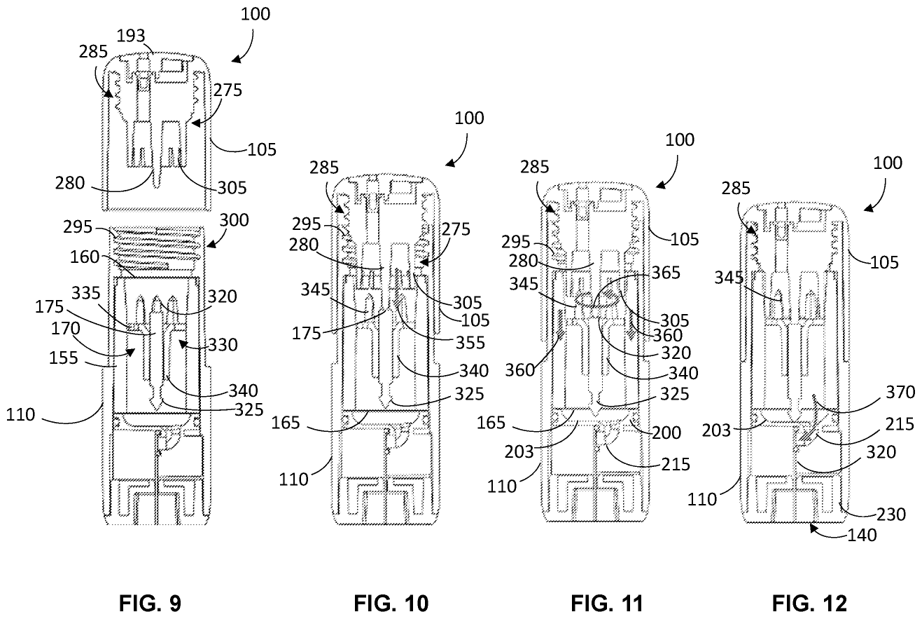

Activation of the device 100 is illustrated in FIGS. 9-12. FIG. 9 shows the device 100 in an inactive state, where the cap 105 is separate from the collar 110. As described above, a user of the device 100 (e.g., a food consumer) may insert a solid or liquid good (e.g., food) into the receptacle 300 of the collar 110. The cap 105 may then be coupled to the collar 110 by engaging the threads 285 with the threads 295, as shown in FIG. 10. As the cap 105 is being coupled to the collar 110, the piercer member 280 pierces the frangible barrier 160 as the piercer member 280 moves in a direction 355, which allows the contents of the receptacle 300 to pass into the first chamber 155. As the cap 105 is turned relative to the collar 110 to engage the threads 285, 295, the grinding assembly 275 and the grinding assembly 170 grind the good from within the receptacle 300 and the ground good then flows into the chamber 155 through the apertures 347 of the support member 330 in a direction 360. The good (e.g., food) within the chamber 155 is then mixed with the buffer solution within the chamber 155. As the cap 105 continues to be threaded onto the collar 110 and the grinding assembly 275 concentrically moves relative to the grinding assembly 170 in a direction 365, and the piercer member 280 contacts and applies an axial force to the piercer member 175, which causes the second end 325 of the piercer member 170 to pierce the frangible barrier 165. Once the frangible barrier 165 is pierced, as shown in FIG. 12, the mixture of the good with the buffer solution from the chamber 155 may then flow into the repository 200 in a direction 370 through the fluid pathway 215. The mixture may then be received by the electrode 220, which may then detect a presence and/or amount of the one or more molecules (i.e., ingredients) within the good.

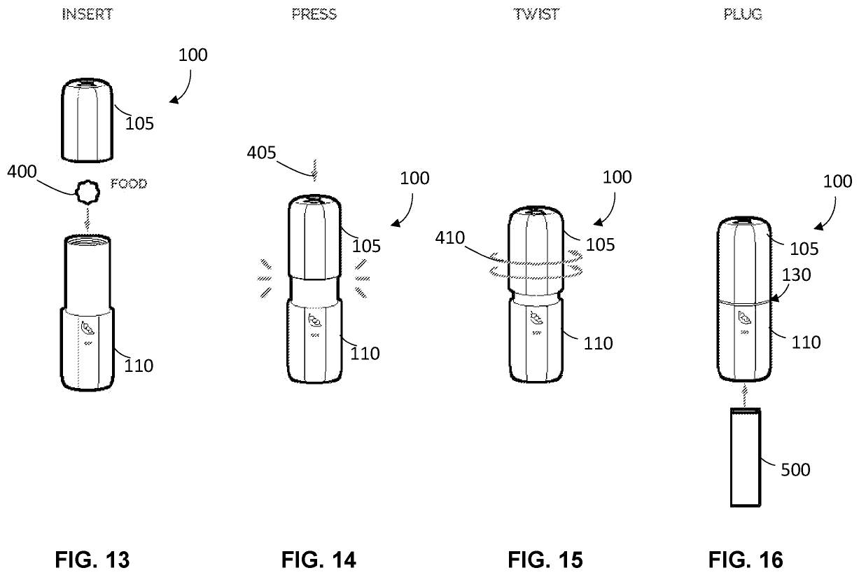

Accordingly, the device 100 may be activated in response to a single user action: coupling the cap 105 to the collar 110. For example, if a user wants to determine if an allergen, toxin, pathogen is present in a food portion 400, the user may insert the good (e.g., food portion) 400 into the collar 110 of the device 100 (FIG. 13). The user may then subsequently press the cap 105 in a direction 405 into the collar 110, as shown in FIG. 14, and rotate the cap in a direction 410 relative to the collar 110, as shown in FIG. 15. The user may then insert an analysis unit 500 into the receiving port 140 of the device 100. In various embodiments, the analysis unit 500 includes or may be in communication with at least one controller. The analysis unit 500 may include one or more processors and a non-transitory computer readable medium (e.g., a memory device) having computer-readable instructions stored thereon that, when executed by a processor, cause the at least one controller to carry out the operations called for by the instructions. In various embodiments, the at least one controller may be a computing device. In other embodiments, the at least one controller may be configured as part of a data cloud computing system configured to receive commands from a user control device and/or remote computing device. When the analysis unit 500 is inserted within the receiving port, the analysis unit 500 is communicably coupled to the electrode 220. Accordingly, when the mixture of the good and the buffer solution is received by the electrode 220, the one or more processors within the analysis unit 500 may receive one or more signals from the electrode 220, where the one or more signals may indicate the presence or an amount (e.g., weight, volume, trace amounts) of the one or more molecules (e.g., ingredient, food ingredient, toxin, allergen, pathogen, etc.) within the good (e.g., food sample). In various embodiments, the analysis unit 500 includes or may be in communication with one or more user interfaces (e.g., graphical user interfaces) configured to indicate to the user of the device 100 that the one or more molecules have been detected or that the one or more molecules have not been detected. In various embodiments, the analysis unit 500 may include one or more visual indicators (e.g., a light or illuminated region) that displays or otherwise indicates that the one or more molecules have been detected or not detected. In yet other embodiments, the analysis unit 500 may be configured to provide audio and/or haptic feedback to the user based on a determination by the one or more processors that the one or more molecules have been detected or not detected.

Notwithstanding the embodiments described above in reference to FIGS. 1-16, various modifications and inclusions to those embodiments are contemplated and considered within the scope of the present disclosure.

As utilized herein with respect to numerical ranges, the terms “approximately,” “about,” “substantially,” and similar terms generally mean +/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

1. A device for detecting an ingredient in a food sample, the device comprising:
a cap comprising a first piercer member; and
a collar being configured to receive the food sample and configured to couple to the cap, wherein the collar comprises:
a first frangible barrier extending across a first cross section of the collar; and
a grinding assembly disposed between the first frangible barrier and a second frangible barrier, the grinding assembly comprising a second piercer member, wherein the second frangible barrier extends across a second cross section of the collar at distance below the first cross section; and
a sensor disposed below the second frangible barrier;
wherein:
a first chamber is formed between the first frangible barrier and the second frangible barrier, the first chamber comprising a volume of a buffer solution;
a second chamber is formed between the second frangible barrier and the sensor;
in response to the cap being coupled to the collar, the first piercer member is configured to pierce the first frangible barrier and the second piercer member is configured to pierce the second frangible barrier;
in response to the each of the first and second frangible barriers being pierced, the first chamber becomes fluidly coupled to the second chamber; and
the food sample received by the collar passes through each of the first chamber and the second chamber to be received by the sensor.

2. The device of claim 1, wherein the collar is threadably coupled to the cap.

3. The device of claim 1, wherein the grinding assembly is disposed above the first chamber, and wherein the food sample mixes with the buffer solution within the first chamber.

4. The device of claim 3, wherein the grinding assembly comprises a support member, the support member configured to concentrically fit within the first chamber such that an outer perimeter defined by an upper surface of the support member is adjacent an inner surface of the first chamber.

5. The device of claim 4, wherein the support member forms a boss, the boss being centrally disposed within the support member and aligned with a longitudinal axis of the device, wherein the boss comprises a bore defined therein, and wherein the second piercer member is configured to fit within and articulate relative to the bore of the central boss.

6. The device of claim 5, wherein the boss projects downward toward a bottom surface of the device and away from the upper surface of the support member.

7. The device of claim 4, wherein the grinding assembly comprises at least two spikes, the at least two spikes projecting upward from the upper surface of the support member.

8. The device of claim 4, wherein the support member comprises a plurality of apertures disposed within the upper surface;
wherein the collar comprises a receptacle configured to receive the food sample, the receptacle being defined between an upper edge of the collar and the first frangible barrier; and
wherein the plurality of apertures enable fluid communication between the receptacle and the first chamber.

9. The device of claim 1, wherein the first piercer member extends from a surface within the cap toward the collar.

10. The device of claim 1, wherein the first piercer member has a first axis and the second piercer has a second axis, and wherein the first axis is aligned with the second axis when the cap is coupled to the collar.

11. The device of claim 10, wherein the second piercer member has a first end and a second end opposite the first end, the first end extending toward the first frangible barrier and the second end extending toward the second frangible barrier, wherein the first piercer member is configured to contact the first end of the second piercer member upon piercing the first foil, and wherein the first piercer member contacting the second piercer member causes the second piercer member to pierce the second frangible barrier.

12. The device of claim 11, wherein the second piercer member slides within the first chamber to pierce the second frangible barrier responsive to the first piercer member contacting the first end of the second piercer member.

13. A system for detecting an ingredient in a food sample, the system comprising:
a sampler unit configured to receive the food sample wherein the sampler unit comprises:
a first piercer member;
a first frangible barrier extending across a first cross section of the sampler unit;
a first grinding assembly disposed between the first frangible barrier and a second frangible barrier, the first grinding assembly comprising a second piercer member, wherein the second frangible barrier extends across a second cross section of the sampler unit at distance below the first cross section; and
an sensor disposed below the second frangible barrier; and
an analysis unit configured to be received within a portion of the sampler unit, the analysis unit comprising a processor, wherein the processor is communicatively couplable to the sensor;
wherein:
a first chamber is formed between the first frangible barrier and the second frangible barrier;
a second chamber is formed between the second frangible barrier and the sensor;
the first piercer member is configured to pierce the first frangible barrier and the second piercer member is configured to pierce the second frangible barrier;
in response to the each of the first and second frangible barriers being pierced, the first chamber becomes fluidly coupled to the second chamber;
the food sample received within the sampler unit passes through each of the first chamber and the second chamber to be received by the sensor; and
the processor is configured to determine a presence of the ingredient in the food sample.

14. The system of claim 13, wherein the second piercer member comprises a first end and a second end opposite the first end, and wherein the first piercer member is configured to contact the first end of the second piercer member when the sampler unit is assembled.

15. The system of claim 14, wherein the second piercer member is configured to articulate within the grinding mechanism responsive to being contacted by the first piercer member such the second piercer moves from a first position to a second position, wherein the second end of the piercer is disposed above the second frangible barrier when in the first position, and wherein the second end of the piercer extends below the second cross-section.

16. The system of claim 13, wherein the sampler unit comprises:
a cap; and
a collar couplable to the cap;
wherein the cap comprises the first piercer member and the collar comprises the second piercer member.

17. The system of claim 16, wherein the cap further comprises a second grinding assembly, and wherein the second grinding assembly is configured to engage with the first grinding assembly when the collar is coupled to the cap.

18. A method for detecting an ingredient in a food sample, the method comprising:
placing the food sample within a collar;
coupling the collar to a cap, wherein:
a first piercer member disposed within the cap pierces a first frangible barrier within the collar responsive to coupling the collar to the cap, and
a second piercer member axially stacked with the first piercer member is configured to pierce a second frangible barrier within the collar responsive to the first piercer member piercing the first frangible barrier;
coupling an analysis unit to the collar by inserting an end of the analysis unit within a recess of the collar, wherein the analysis unit comprises a processor;
wherein:
the processor is communicatively couplable to a sensor disposed within the collar, and
the sensor is in fluid communication with at least a portion of the food sample responsive to the second piercer member piercing the second frangible barrier; and
determining a presence of the ingredient in the food sample based on an output by the processor within the analysis unit.

19. The method of claim 18, wherein the ingredient comprises a food ingredient.

20. The method of claim 19, wherein the food ingredient comprises at least one of an allergen, food toxin, or food pathogen.

21. The method of claim 19, wherein the sensor is an electrode.
